(19) United States
(12) Patent Application Publication
(10) Pub. No.: US 2025/0325722 A1
(43) Pub. Date: Oct. 23, 2025

(54) Method of Deodorizing, Antibacterial and Decontamination Using Plasmonic Photocatalyst and Method for Treating Cancer

(71) Applicant: National Tsing Hua University, Hsinchu (TW)

(72) Inventor: Kuo Chu Hwang, Hsinchu (TW)

(21) Appl. No.: 18/815,078

(22) Filed: Aug. 26, 2024

(30) Foreign Application Priority Data
Apr. 22, 2024 (TW) 113114841

(51) Int. Cl. A61L 2/23; A61K 33/244; A61K 45/06; A61L 2/00

(52) U.S. Cl. CPC A61L 2/23; A61K 33/244; A61K 45/06; A61L 2/0047

(57) ABSTRACT

A method of deodorizing, antibacterial and decontamination using a plasmonic photocatalyst includes the steps as follows. A plasmonic photocatalyst is provided. The plasmonic photocatalyst and an object to be treated are made to be in contact. The plasmonic photocatalyst is made to absorb an electromagnetic wave to perform a deodorizing, antibacterial and decontamination reaction on the object to be treated, and the deodorizing, antibacterial and decontamination reaction is to perform an oxidative decomposition or removal reaction on a contaminant in the object to be treated. A wavelength of the electromagnetic wave is in a range of 300 nm to 11000 nm. A method of treating cancer is also disclosed.

### CROSS-REFERENCE TO RELATED APPLICATION

This application claims priority to Taiwanese Patent Application No. 113114841, filed Apr. 22, 2024, the disclosure of which is hereby incorporated by reference in its entirety.

### BACKGROUND OF THE INVENTION

### Field of the Invention

The present disclosure relates to a method of deodorizing, antibacterial and decontamination and a use of a pharmaceutical composition. More particularly, the present disclosure relates to a method of deodorizing, antibacterial and decontamination using a plasmonic photocatalyst and a use of a pharmaceutical composition for treating cancer.

### Description of Related Art

Photocatalyst is a material which can effectively remove harmful substances in the air or the environment under light irradiation. The mechanism thereof is to absorb ultraviolet light, visible light or near infrared light by photocatalyst nanoparticles with a wavelength shorter than 1000 nm, and then convert the nearby oxygen molecules or water molecules into the hydroxyl radicals with high activity to catalyze and degrade the harmful substances. For example, photocatalyst nanoparticles are often used to remove volatile organic compounds and nitrogen oxides from the air, or organic pollutants from water.

The first step for photocatalyst nanoparticles to function is to absorb ultraviolet light, visible light or near infrared light with the wavelength shorter than 1000 nm in order to successfully perform photocatalytic decomposition of pollutants. The source of ultraviolet light, visible light or near infrared light sources has to be provided externally, or directly from sunlight. The requirement of an external light source to perform photocatalytic decomposition very often limits the applications of photocatalysts. Also, it is difficult to perform photocatalytic decomposition in public areas where the space is very large or cannot be regularly maintained, such as train/bus stations, restaurants, or ATMs. Moreover, most photocatalysts cannot function under the conditions where there is no light, at night or lack of external light sources.

In this regard, it is a goal for relevant industry to reduce the reliance on external light sources and reduce the restrictions as using photocatalysts.

On the other hand, in the treatment of tumors and cancers using near infrared photodynamic therapy and near infrared photothermal therapy, inorganic nanoparticles are mostly used as photosensitizers to absorb near infrared light in the conventional techniques to generate the reactive oxygen species (ROS) or heat, and to inhibit the growths of tumor cells. The wavelength of near infrared light used therein is usually between 800 nm to 1550 nm. Because the wavelength of the near infrared light used in the conventional techniques is not long enough, their penetration depths into biological tissues are very limited, which restricts their applications in treating deeply-seated tumors or results in poor treatment efficacies.

Accordingly, if a nanomaterial particle which can be activated by near infrared light with a wavelength longer than 1550 nm to generate the reactive oxygen species or the heat can be developed, it will break through the disadvantages of the conventional near infrared photodynamic therapy and photothermal therapy not able to treat deeply-seated tumors.

### SUMMARY OF THE INVENTION

According to one embodiment of the present disclosure, a method of deodorizing, antibacterial and decontamination using a plasmonic photocatalyst includes the steps as follows. A plasmonic photocatalyst is provided. The plasmonic photocatalyst and an object to be treated are made to be in contact. The plasmonic photocatalyst is made to absorb an electromagnetic wave to perform a deodorizing, antibacterial and decontamination reaction on the object to be treated, and the deodorizing, antibacterial and decontamination reaction is to perform an oxidative decomposition or removal reaction on a contaminant in the object to be treated. A wavelength of the electromagnetic wave is in a range of 300 nm to 11000 nm.

According to another embodiment of the present disclosure, a method for treating cancer includes administering a pharmaceutical composition including a plasmonic photocatalyst to a subject in need for a treatment of cancer. After administering the pharmaceutical composition, the plasmonic photocatalyst absorbs an electromagnetic wave, and a wavelength of the electromagnetic wave is in a range of 300 nm to 11000 nm.

### BRIEF DESCRIPTION OF THE DRAWINGS

The present disclosure can be more fully understood by reading the following detailed description of the embodiment, with reference made to the accompanying drawings as follows:

FIG. 1 is a step flow chart of a method of deodorizing, antibacterial and decontamination using a plasmonic photocatalyst according to an embodiment of the present disclosure.

FIG. 2A is an electron paramagnetic resonance spectrogram of the photocatalyst-containing aqueous solution under different irradiation time of the 1^{st} example.

FIG. 2B is an electron paramagnetic resonance spectrogram of the photocatalyst-containing aqueous solution receiving different wavelengths of irradiation of the 1^{st} example.

FIG. 3A is a fluorescence intensity diagram of the photocatalyst-containing aqueous solution under different irradiation time of the 2^{nd} example.

FIG. 3B is another fluorescence intensity diagram of the photocatalyst-containing aqueous solution under different irradiation time of the 2^{nd} example.

FIG. 4 is an absorption result diagram of the photocatalyst-containing aqueous solution under different irradiation time of the 3^{rd} example.

FIG. 5 is a comparison diagram of dye concentration under different irradiation time of the photocatalyst-containing aqueous solutions of the 3^{rd} example, the 2^{nd} comparative example and the 3^{rd} comparative example and the aqueous solution of the 1^{st} comparative example.

FIG. 6 is a comparison diagram of viability of E. coli under different irradiation time of the photocatalyst-containing aqueous solutions of the 4^{th} example to the 6^{th} example and the aqueous solution of the 1^{st} comparative example.

FIG. 7A is a microscopic image of the photocatalyst-containing aqueous solution before irradiation of the 5^{th} example.

FIG. 7B is a microscopic image of the photocatalyst-containing aqueous solution after irradiation of the 5^{th} example.

### DESCRIPTION OF THE INVENTION

The present disclosure will be further exemplified by the following specific embodiments. The embodiments can be applied to various inventive concepts and can be embodied in various specific ranges. The specific embodiments are only for the purposes of description, and are not limited to these practical details thereof.

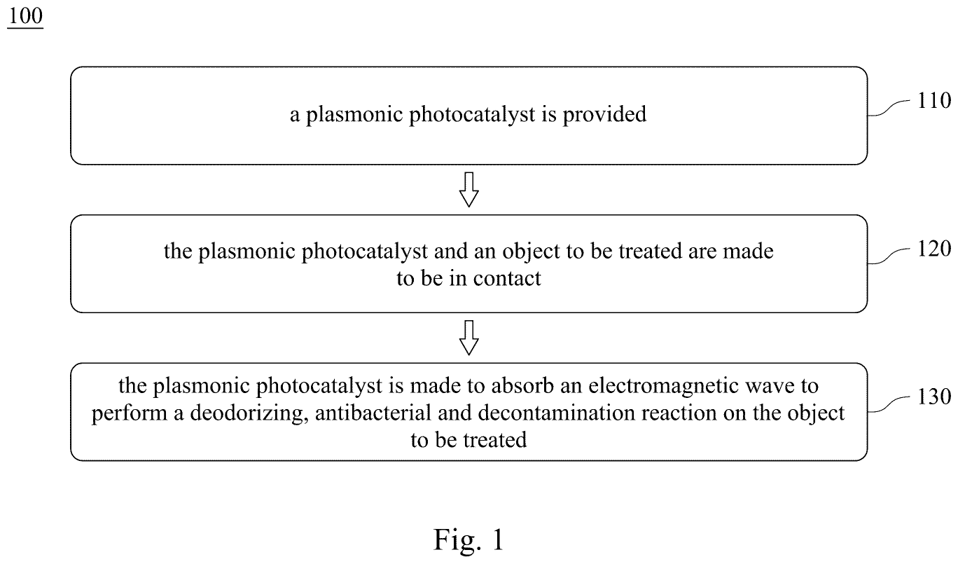

Reference is made to FIG. 1. FIG. 1 is a step flow chart of a method of deodorizing, antibacterial and decontamination using a plasmonic photocatalyst 100 according to an embodiment of the present disclosure. The method of deodorizing, antibacterial and decontamination using the plasmonic photocatalyst 100 includes Step 110, Step 120 and Step 130.

In detail, Step 110 is to provide a plasmonic photocatalyst. The plasmonic photocatalyst can be a metal boride nanoparticle, a metal nitride nanoparticle or a combination thereof. The metal boride nanoparticle can be a lanthanum hexaboride (LaB₆) nanoparticle, and a particle diameter of the lanthanum hexaboride nanoparticle can be within 5 nm to 300 nm.

Moreover, the plasmonic photocatalyst can be a photocatalyst-containing aqueous solution, that is, the plasmonic photocatalyst can be suspended in an aqueous solution. The photocatalyst-containing aqueous solution can include at least one of the metal boride nanoparticle and the metal nitride nanoparticle. The metal element can be a transition metal element or a lanthanide metal element. The photocatalyst-containing aqueous solution preferably includes the lanthanum hexaboride nanoparticle. An amount of the lanthanum hexaboride nanoparticle in the photocatalyst-containing aqueous solution can range from 10 μg/mL to 1000 μg/mL. Moreover, the ratio of the lanthanum hexaboride nanoparticle in the photocatalyst-containing aqueous solution can be 25 μg/mL to 800 μg/mL. Moreover, the ratio of the lanthanum hexaboride nanoparticle in the photocatalyst-containing aqueous solution can be 50 μg/mL to 500 μg/mL. Moreover, the ratio of the lanthanum hexaboride nanoparticle in the photocatalyst-containing aqueous solution can be 100 μg/mL to 200 μg/mL. It can be understood that, the plasmonic photocatalyst of the present disclosure can be different types for different processes and application conditions. Therefore, the present disclosure is not limited to the type of the plasmonic photocatalyst.

Step 120 is to make the plasmonic photocatalyst and an object to be treated stay in contact, wherein the object to be treated can be gas, liquid or solid. Also, the type of the plasmonic photocatalyst can be adjusted according to the type of the object to be treated. For example, when the object to be treated is solid, the plasmonic photocatalyst can be the aforementioned photocatalyst-containing aqueous solution, and can be in contact with the object to be treated by the methods such as wiping or soaking. Furthermore, the object to be treated can include a contaminant, and the contaminant can be odor molecules or bacteria. However, the present disclosure is not limited to the types of the object to be treated and the contaminant.

Step 130 is to make the plasmonic photocatalyst absorb an electromagnetic wave to perform a deodorizing, antibacterial and decontamination reaction on the object to be treated, and the deodorizing, antibacterial and decontamination reaction is to perform an oxidative decomposition or removal reaction on the contaminant in the object to be treated. A wavelength of the electromagnetic wave is in a range of 300 nm to 11000 nm, which provides sufficient energy for the plasmonic photocatalyst to perform the deodorizing, antibacterial and decontamination reaction. The plasmonic photocatalyst can keep absorbing the electromagnetic wave until a pre-set reaction time, and the pre-set reaction time can range from 1 minute to 5 years to enhance the performance of the deodorizing, antibacterial and decontamination reaction. The plasmonic photocatalyst can perform the aforementioned reactions immediately upon absorbing the electromagnetic wave and the technique effects of the present disclosure can be achieved instantly, so the present disclosure is not limited to the aforementioned pre-set reaction time.

For example, in the deodorizing, antibacterial and decontamination reaction, the plasmonic photocatalyst can react with a water molecule to generate a hydroxyl radical under irradiation of the electromagnetic wave, and the hydroxyl radical can perform the oxidative decomposition or removal reaction on the contaminant in the object to be treated. It should be mentioned that, the reaction mechanism of the plasmonic photocatalyst is related to the material types included therein, so the present disclosure is not limited to the aforementioned reaction mechanism.

The wavelength of the electromagnetic wave can range from 1550 nm to 11000 nm. Therefore, the plasmonic photocatalyst can absorb near infrared light or mid-infrared light to perform the deodorizing, antibacterial and decontamination reaction.

According to another embodiment of the present disclosure, a method for treating cancer includes administration of a pharmaceutical composition including a plasmonic photocatalyst to a subject in need for a treatment of cancer. After administration of the pharmaceutical composition, the plasmonic photocatalyst absorbs an electromagnetic wave, and a wavelength of the electromagnetic wave is in a range of 300 nm to 11000 nm.

The plasmonic photocatalyst can be a metal boride nanoparticle or a metal nitride nanoparticle, and the metal boride nanoparticle or the metal nitride nanoparticle absorbs the electromagnetic wave and generates at least one of a reactive oxygen species and a heat. In detail, the metal boride nanoparticle or the metal nitride nanoparticle can absorb long-wavelength mid-infrared light (with the wavelength between 1550 nm to 11000 nm) which can effectively penetrate biological tissues, and generate the reactive oxygen species or heat to kill cancer cells and inhibit the growths of tumors.

If the surface of the metal boride nanoparticle or the metal nitride nanoparticle is chemical-modified to target specific molecular receptors on the surface of specific tumor cells, the plasmonic photocatalyst which is surface-modified with the specific biomarker probes can circulate in the blood in a living body. It has the specificity to bind to the receptors on the cell membrane surfaces of the specific tumor cells, and can actively target the tumor tissues and accumulate in the tumor tissues. Through the irradiation of long-wavelength mid-infrared light outside the body, the reactive oxygen species or heat will be generated within the tumor tissues to inhibit the growths of tumor cells around the plasmonic photocatalyst.

Moreover, the pharmaceutical composition can further include an antibody, and the antibody can be an epidermal growth factor receptor (EGFR) antibody, a vascular endothelial growth factor receptor (VEGFR) antibody, a T-cell receptor-like (TCR-like) antibody or an antibody of a specific receptor on the carcinoid cell surface. Moreover, the pharmaceutical composition can further include a biological probe, and the biological probe can include a folic acid, a cell-penetrating TAT peptide, a fibroblast activation protein inhibitor (FAPI), an arginylglycylaspartic acid (RGD) peptide or a heparin (HEP) polysaccharide. However, the present disclosure is not limited to the aforementioned antibody or the aforementioned biological probe.

Accordingly, the metal boride nanoparticle or the metal nitride nanoparticle can inhibit the growths of tumor cells. Also, through the surface-modification of the antibody or the biological probe, it can be used as the photosensitizers of near infrared or mid-infrared photodynamic therapy and photothermal therapy with active tumor targeting function. After irradiation of near infrared light or mid-infrared light, the reactive oxygen species or heat will be generated to inhibit the growths of deeply-seated tumor cells. Furthermore, the problem of the conventional photodynamic therapy and photothermal therapy lacking photosensitizers which can absorb long-wavelength near infrared light and/or mid-infrared light to generate active oxygen-containing free radicals and the heat can be solved.

The tumor cells or tumor tissues described in the present disclosure include, but are not limited to, melanoma, brain tumors, lung tumors or head and neck tumors. Also, the surface of the metal boride nanoparticle or the metal nitride nanoparticle can be modified with different antibodies or biological probes to actively target other types of tumor tissues.

The present disclosure will be further exemplified by the following specific embodiments so as to facilitate utilizing and practicing the present disclosure completely by the people skilled in the art without over-interpreting and over-experimenting. However, the readers should understand that the present disclosure should not be limited to these practical details thereof, that is, these practical details are used to describe how to implement the materials and methods of the present disclosure and are not necessary.

### EXAMPLES AND COMPARATIVE EXAMPLES

### 1^{st} Example

The plasmonic photocatalyst used in the method of deodorizing, antibacterial and decontamination using the plasmonic photocatalyst of the 1^{st} example is a photocatalyst-containing aqueous solution, and the photocatalyst-containing aqueous solution includes a lanthanum hexaboride nanoparticle. A ratio of the lanthanum hexaboride nanoparticle in the photocatalyst-containing aqueous solution is 100 μg/mL.

### 2^{nd} Example

The method of deodorizing, antibacterial and decontamination using the plasmonic photocatalyst of the 2^{nd} example is similar to the 1^{st} example. However, in the 2^{nd} example, a ratio of the lanthanum hexaboride nanoparticle in the photocatalyst-containing aqueous solution is 500 μg/mL.

### 3^{rd} Example

The method of deodorizing, antibacterial and decontamination using the plasmonic photocatalyst of the 3^{rd} example is similar to the 1^{st} example. However, in the 3^{rd} example, a ratio of the lanthanum hexaboride nanoparticle in the photocatalyst-containing aqueous solution is 200 μg/mL.

### 4^{th} Example

The method of deodorizing, antibacterial and decontamination using the plasmonic photocatalyst of the 4^{th} example is similar to the 1^{st} example. However, in the 4^{th} example, a ratio of the lanthanum hexaboride nanoparticle in the photocatalyst-containing aqueous solution is 25 μg/mL.

### 5^{th} Example

The method of deodorizing, antibacterial and decontamination using the plasmonic photocatalyst of the 5^{th} example is similar to the 1^{st} example. However, in the 5^{th} example, a ratio of the lanthanum hexaboride nanoparticle in the photocatalyst-containing aqueous solution is 50 μg/mL.

### 6^{th} Example

The method of deodorizing, antibacterial and decontamination using the plasmonic photocatalyst of the 6^{th} example is similar to the 1^{st} example. However, in the 6^{th} example, a ratio of the lanthanum hexaboride nanoparticle in the photocatalyst-containing aqueous solution is 50 μg/mL. Moreover, the photocatalyst-containing aqueous solution of the 6^{th} example further includes ascorbic acid (AA).

### 7^{th} Example

The method for treating cancer of the 7^{th} example includes administering a pharmaceutical composition including a plasmonic photocatalyst, and the plasmonic photocatalyst includes a lanthanum hexaboride nanoparticle.

### 1^{st} Comparative Example

In the method of the 1^{st} comparative example, an aqueous solution and an object to be treated are made to be in contact with the plasmonic photocatalysts. Then, the aqueous solution is made to absorb the electromagnetic wave to perform an oxidative decomposition or removal reaction on a contaminant in the object to be treated. The aqueous solution of the 1^{st} comparative example does not include any plasmonic photocatalyst.

### 2^{nd} Comparative Example

In the method of the 2^{nd} comparative example, a photocatalyst-containing aqueous solution and an object to be treated are made to be in contact. Then, the photocatalyst-containing aqueous solution is made to absorb the electromagnetic wave to perform an oxidative decomposition or removal reaction on a contaminant in the object to be treated. The photocatalyst-containing aqueous solution of the 2^{nd} comparative example includes titanium dioxide nanoparticles, and a ratio of the titanium dioxide nanoparticles in the photocatalyst-containing aqueous solution is 1000 μg/mL.

### 3^{rd} Comparative Example

The method of the 3^{rd} comparative example is similar to the 2^{nd} comparative example. However, the photocatalyst-containing aqueous solution of the 3^{rd} comparative example includes gold nanoparticles, and a ratio of the gold nanoparticles in the photocatalyst-containing aqueous solution is 200 μg/mL.

### Experiment of Activated Effects by Electromagnetic Wave

In the present experiment, 100 mM DMPO (5,5-dimethyl-1-pyrroline N-oxide) was added into the photocatalyst-containing aqueous solution of the 1^{st} example. LEDs with different wavelengths were used as the source of the electromagnetic wave to irradiate the photocatalyst-containing aqueous solution of the 1^{st} example. Then, electron paramagnetic resonance signals were collected by the electron paramagnetic resonance spectrometer for analysis.

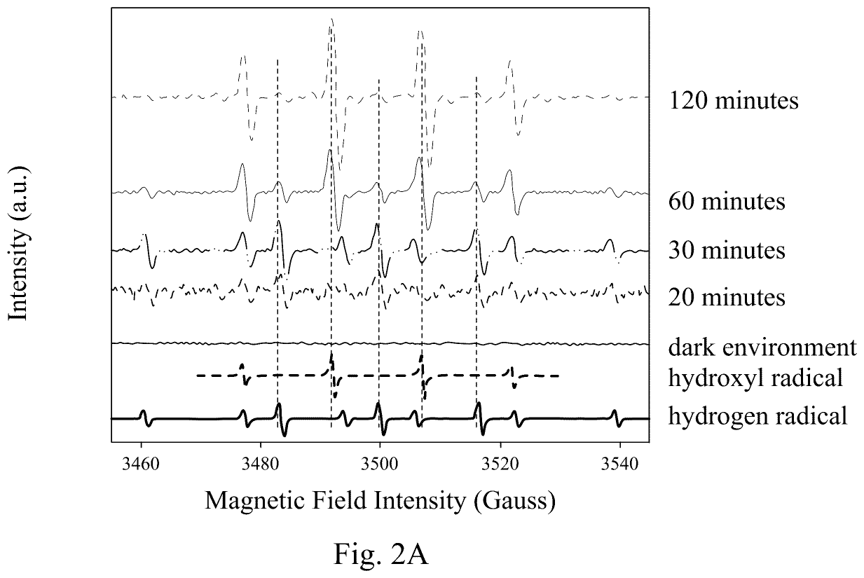

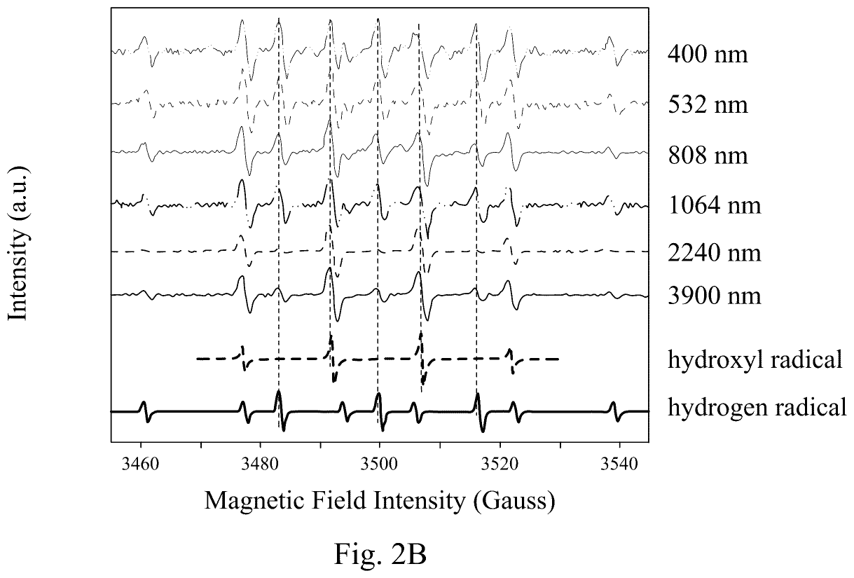

Reference is made to FIG. 2A. FIG. 2A is an electron paramagnetic resonance spectrogram of the photocatalyst-containing aqueous solution under different irradiation time of the 1^{st} example. FIG. 2A was obtained by collecting the electron paramagnetic resonance signals of LED light with the wavelength of 3900 nm irradiating the photocatalyst-containing aqueous solution of the 1^{st} example for different irradiation time. In FIG. 2A, compared to that without irradiation (i.e., dark environment), the photocatalyst-containing aqueous solution including the lanthanum hexaboride nanoparticle can generate the hydroxyl radical and the hydrogen radical under light irradiation no matter how long the time is.

Reference is made to FIG. 2B. FIG. 2B is an electron paramagnetic resonance spectrogram of the photocatalyst-containing aqueous solution receiving different wavelengths of irradiation of the 1^{st} example. FIG. 2B was obtained by collecting the electron paramagnetic resonance signals of LED lights with different wavelengths irradiating the photocatalyst-containing aqueous solution of the 1^{st} example for 60 minutes. In FIG. 2B, the photocatalyst-containing aqueous solution including the lanthanum hexaboride nanoparticle can generate the hydroxyl radical and the hydrogen radical under light irradiation with different wavelengths.

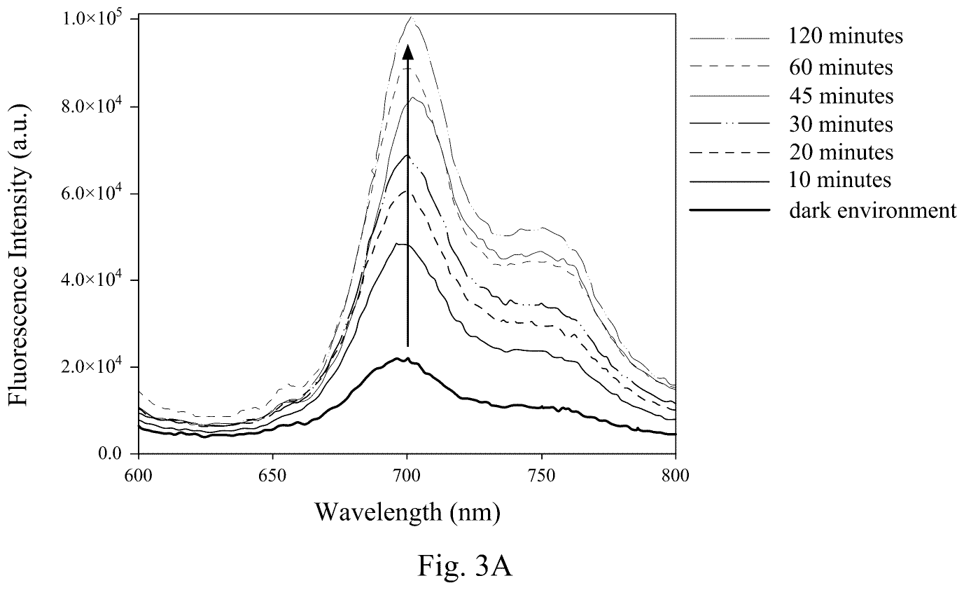

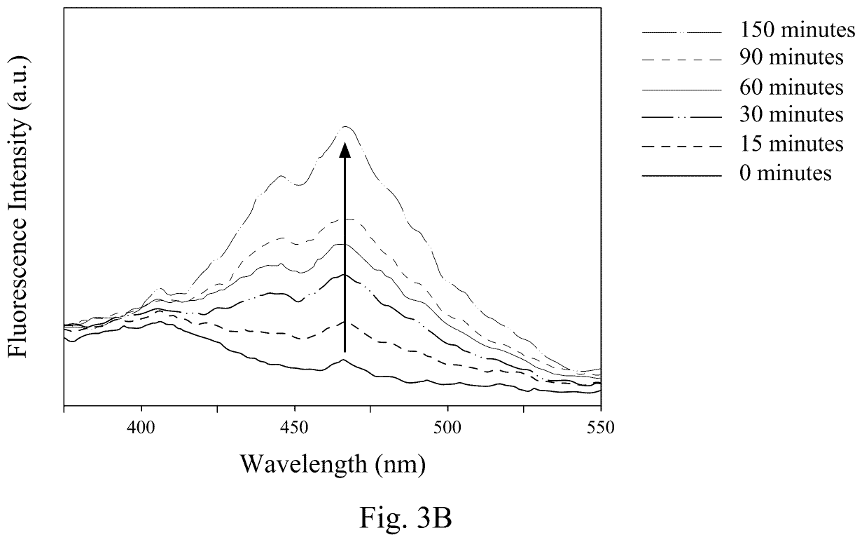

Reference is made to FIG. 3A and FIG. 3B. FIG. 3A is a fluorescence intensity diagram of the photocatalyst-containing aqueous solution under different irradiation time of the 2^{nd} example. FIG. 3B is another fluorescence intensity diagram of the photocatalyst-containing aqueous solution under different irradiation time of the 2nd example. In FIG. 3A, probe 2b was added into the photocatalyst-containing aqueous solution of the 2^{nd} example. The probe 2b is a specific fluorescent probe for the hydroxyl radical. Then, the fluorescence intensity of the photocatalyst-containing aqueous solution of the 2^{nd} example irradiated by the LED light with the wavelength of 3900 nm for different irradiation time was measured. If the fluorescence intensity is stronger, the concentration of the hydroxyl radical is higher. In FIG. 3B, coumarin was added into the photocatalyst-containing aqueous solution of the 2^{nd} example. Coumarin reacted with the hydroxyl radical to generate umbelliferone which is fluorescent under UV light irradiation. Then, the fluorescence intensity of the photocatalyst-containing aqueous solution of the 2^{nd} example irradiated by the LED light with the wavelength of 3900 nm for different irradiation time was measured. If the fluorescence intensity is stronger, the concentration of the hydroxyl radical is higher. The structural formula of the probe 2b is shown in the following figure:

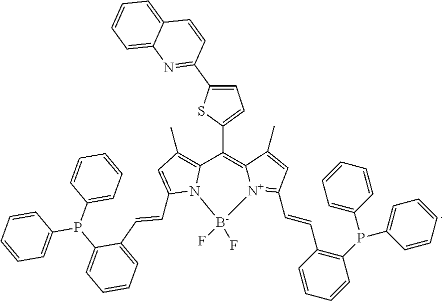 [SMILES: CC1=CC(/C=C/c2ccccc2P(c2ccccc2)c2ccccc2)=[N+]2C1=C(c1ccc(-c3ccc4ccccc4n3)s1)c1c(C)cc(/C=C/c3ccccc3P(c3ccccc3)c3ccccc3)n1[B-]2(F)F]

In FIG. 3A and FIG. 3B, compared to that without irradiation (dark environment and 0 minutes), the photocatalyst-containing aqueous solution including the lanthanum hexaboride nanoparticle can generate the hydroxyl radical under light irradiation no matter how long the time is, and the concentration of the hydroxyl radical increases as the irradiation time of light source increases.

### <Experiment of Cleaning Effects>

In the present experiment, neutral red dye was added into the photocatalyst-containing aqueous solution of the 3^{rd} example as the contaminant. Then, the absorbance of the photocatalyst-containing aqueous solution of the 3^{rd} example irradiated by the LED light with the wavelength of 3900 nm for different irradiation time was measured. If the absorbance is lower, the decomposition degree of the neutral red dye is higher.

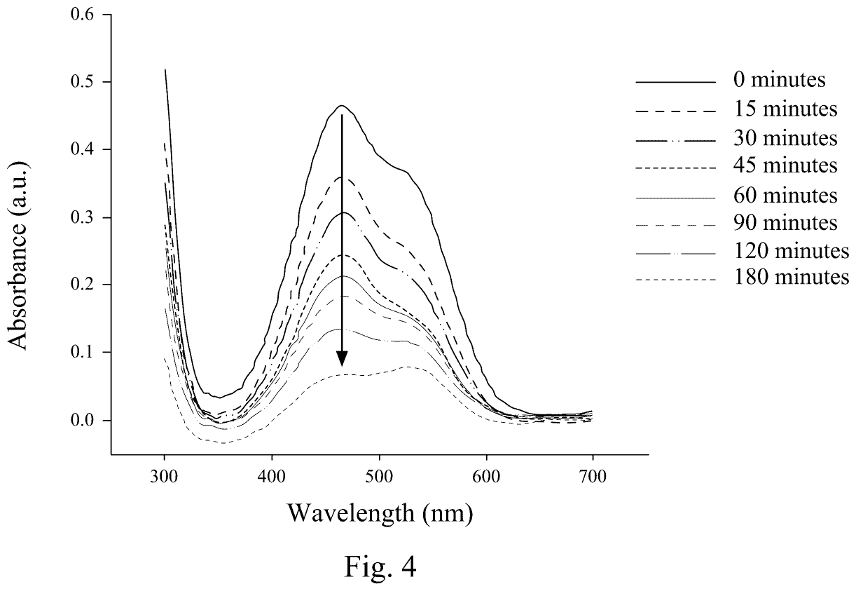

Reference is made to FIG. 4. FIG. 4 is an absorption result diagram of the photocatalyst-containing aqueous solution under different irradiation time of the 3rd example. In FIG. 4, the photocatalyst-containing aqueous solution including the lanthanum hexaboride nanoparticle can decompose the contaminant after irradiated by the light source no matter how long the time is, and the degree of decomposition increases as the irradiation time of light source increases.

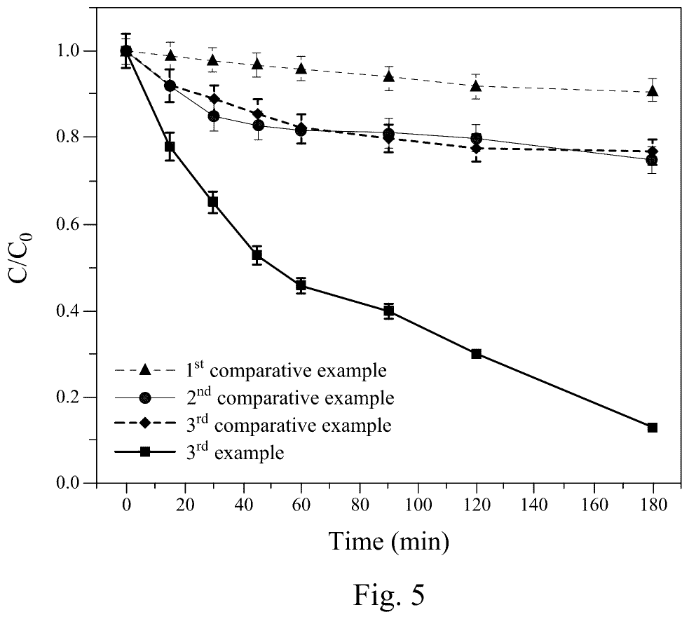

Reference is made to FIG. 5. FIG. 5 is a comparison diagram of dye concentration under different irradiation time of the photocatalyst-containing aqueous solutions of the 3^{rd} example, the 2^{nd} comparative example and the 3^{rd} comparative example and the aqueous solution of the 1^{st} comparative example. In FIG. 5, the neutral red dye was added into the photocatalyst-containing aqueous solutions of the 3^{rd} example, the 2nd comparative example and the 3^{rd} comparative example and the aqueous solution of the 1^{st} comparative example as the contaminant. After light irradiation, the dye concentrations under different irradiation time were calculated, and the dye concentration before irradiation (Co) and the dye concentration after irradiation (C) were compared. In FIG. 5, the decomposition rate of the contaminant in the photocatalyst-containing aqueous solution of the 3^{rd} example is significantly higher than those of the 1^{st} comparative example to the 3^{rd} comparative example, which proves that the photocatalyst-containing aqueous solution including the lanthanum hexaboride nanoparticle has great cleaning ability.

### <Experiment of Antibacterial Effects>

In the present experiment, E. coli (Escherichia coli) was added into the photocatalyst-containing aqueous solutions of the 4^{th} example to the 6^{th} example and the aqueous solution of the 1^{st} comparative example as the contaminant. After the irradiation, the numbers of E. coli colonies under different irradiation time were calculated, and the number of colonies without photocatalyst treatment and light irradiation (Fo) and the number of colonies after irradiation (F) were compared. Furthermore, the photocatalyst-containing aqueous solution of the 4^{th} example without irradiation was taken as a comparison in the present experiment to evaluate the antibacterial effects of other groups.

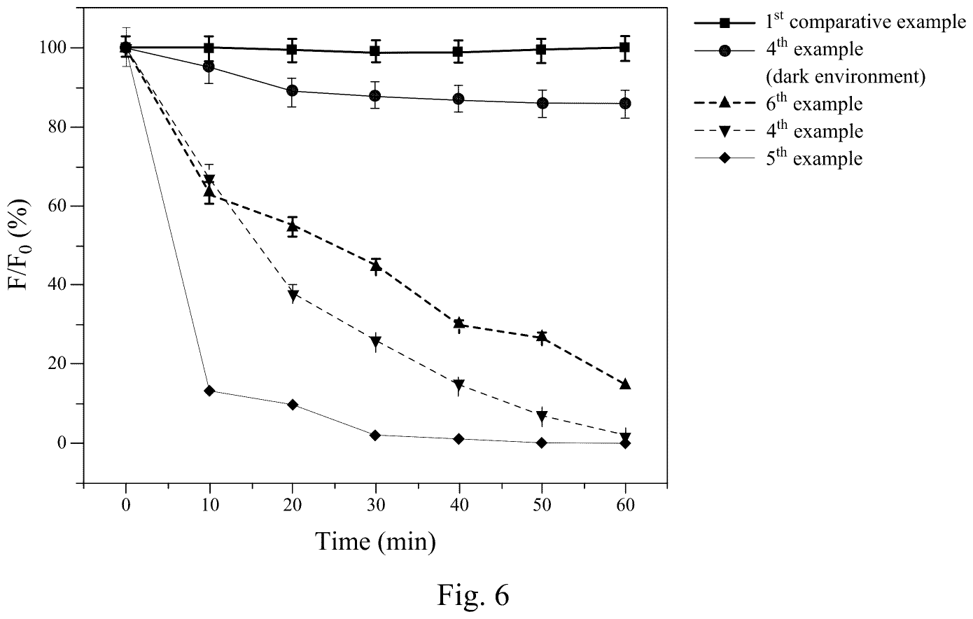

Reference is made to FIG. 6. FIG. 6 is a comparison diagram of viability of E. coli under different irradiation time of the photocatalyst-containing aqueous solutions of the 4^{th} example to the 6^{th} example and the aqueous solution of the 1^{st} comparative example. In FIG. 6, the photocatalyst-containing aqueous solution of the 4^{th} example to the 6th example can effectively reduce the number of E. coli colonies after irradiation, which proves the photocatalyst-containing aqueous solution including the lanthanum hexaboride nanoparticle has great antibacterial effect.

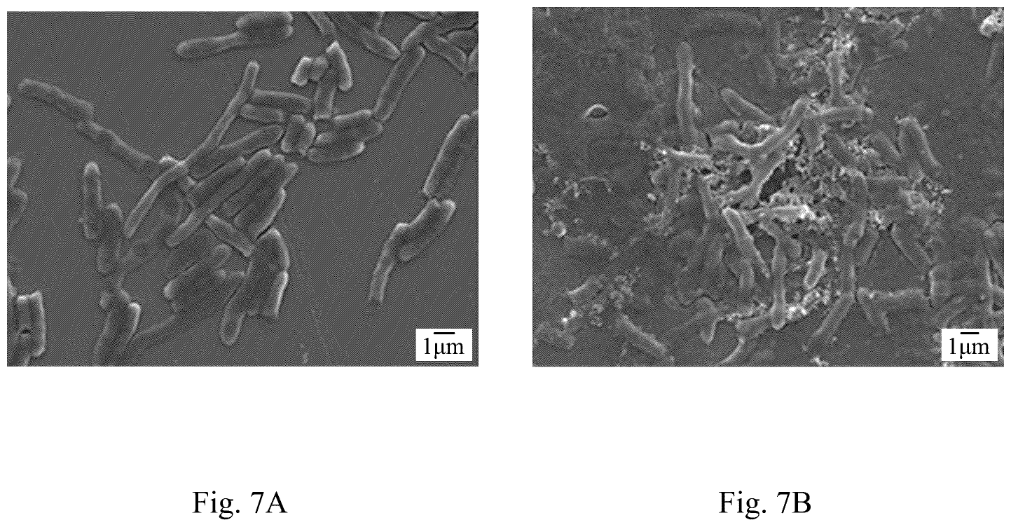

Reference is made to FIG. 7A and FIG. 7B. FIG. 7A is a microscopic image of the photocatalyst-containing aqueous solution before irradiation of the 5^{th} example. FIG. 7B is a microscopic image of the photocatalyst-containing aqueous solution after irradiation of the 5^{th} example. In FIG. 7A and FIG. 7B, the E. coli cells are intact in FIG. 7A, and the E. coli breaks after irradiation and become cell debris as shown in FIG. 7B. It proves that the photocatalyst-containing aqueous solution including the lanthanum hexaboride nanoparticle can indeed achieve the antibacterial effect.

### <Cell Viability>

In the present experiment, NCI-H23 cells with a density of 10⁴ cells/mL were cultured in a 24-well plate for 24 hours. Then, the plasmonic photocatalyst of the 7th example was made to be in contact with the NCI-H23 cells to be co-cultured for 24 hours in dark environment under 37° C., and be irradiated by laser light of 808 nm, 980 nm, 1550 nm and 2240 nm for 16 minutes. After irradiation and culture overnight, 50 μL 3-(4,5-dimethylthiazol-2-yl)-2,5-diphenyltetrazolium bromide (MTT) reagent with a concentration of 0.5 mg/mL was added therein and cultured for another 4 hours. Then, the supernatant was discarded, and 1 mL dimethyl sulfoxide (DMSO) was added into each well to be centrifuged under 13000 rpm. Finally, the supernatant was collected for an ELISA measurement under a wavelength of 570 nm. The measured values of optical absorbance were converted into cell viabilities, and the results thereof are shown in Table 1 below.

TABLE 1

| Wavelength of Laser (nm) | Cell Viability (%) |
|---|---|
| 808 | 39.3 |
| 980 | 31.2 |
| 1550 | 28.2 |
| 2240 | 28.7 |

In Table 1, the method for treating cancer of the present disclosure has the effect of inhibiting the growth of NCI-H23 cells under the laser with different wavelengths. As the wavelength of laser increases, the inhibiting effect thereof becomes more apparent, which proves the method for treating cancer of the present disclosure can effectively inhibit the growth of tumor cells under long-wavelength near infrared light and mid-infrared light.

In this regard, in the method of deodorizing, antibacterial and decontamination using the plasmonic photocatalyst and the method for treating cancer of the present disclosure, the plasmonic photocatalyst used therein can absorb the electromagnetic wave with various wavelengths as the source of energy. The wavelength range of the electromagnetic wave can range from ultraviolet light to mid-infrared light. Therefore, the method of deodorizing, antibacterial and decontamination using the plasmonic photocatalyst and the method for treating cancer of the present disclosure can be performed using the light and heat radiation in the environment. Without applying additional energy or providing the light source, the hydroxyl radical can be generated to perform oxidative-deodorizing, antibacterial and decontamination on the target substance, or the medicinal composition for treating cancer can be manufactured therefrom. Therefore, it has the potential for applications in various fields.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims.

What is claimed is:

1. A method of deodorizing, antibacterial and decontamination using a plasmonic photocatalyst, comprising:
providing a plasmonic photocatalyst;
making the plasmonic photocatalyst and an object to be treated be in contact; and
making the plasmonic photocatalyst absorb an electromagnetic wave to perform a deodorizing, antibacterial and decontamination reaction on the object to be treated, and the deodorizing, antibacterial and decontamination reaction is to perform an oxidative decomposition or removal reaction on a contaminant in the object to be treated;
wherein a wavelength of the electromagnetic wave is in a range of 300 nm to 11000 nm.

2. The method of deodorizing, antibacterial and decontamination using the plasmonic photocatalyst of claim 1, wherein the plasmonic photocatalyst is a metal boride nanoparticle, a metal nitride nanoparticle or a combination thereof.

3. The method of deodorizing, antibacterial and decontamination using the plasmonic photocatalyst of claim 2, wherein the metal boride nanoparticle is a lanthanum hexaboride nanoparticle, and a particle diameter of the lanthanum hexaboride nanoparticle is within 5 nm to 300 nm.

4. The method of deodorizing, antibacterial and decontamination using the plasmonic photocatalyst of claim 1, wherein in the deodorizing, antibacterial and decontamination reaction, the plasmonic photocatalyst reacts with a water molecule to form a hydroxyl radical under irradiation of the electromagnetic wave, and the hydroxyl radical performs the oxidative decomposition or removal reaction on the contaminant in the object to be treated.

5. The method of deodorizing, antibacterial and decontamination using the plasmonic photocatalyst of claim 1, wherein the plasmonic photocatalyst keeps absorbing the electromagnetic wave until a pre-set reaction time, and the pre-set reaction time ranges from 1 minute to 5 years.

6. The method of deodorizing, antibacterial and decontamination using the plasmonic photocatalyst of claim 1, wherein the plasmonic photocatalyst is a photocatalyst-containing aqueous solution, and the photocatalyst-containing aqueous solution comprises a lanthanum hexaboride nanoparticle.

7. A method for treating cancer, comprising administering a pharmaceutical composition comprising a plasmonic photocatalyst to a subject in need for a treatment of cancer;
wherein after administering the pharmaceutical composition, the plasmonic photocatalyst absorbs an electromagnetic wave, and a wavelength of the electromagnetic wave is in a range of 300 nm to 11000 nm.

8. The method for treating cancer of claim 7, wherein the plasmonic photocatalyst is a metal boride nanoparticle, a metal nitride nanoparticle or a combination thereof, and the metal boride nanoparticle or the metal nitride nanoparticle absorbs the electromagnetic wave and generates at least one of a reactive oxygen species and a heat.

9. The method for treating cancer of claim 7, wherein the pharmaceutical composition further comprises an antibody, and the antibody is an epidermal growth factor receptor antibody, a vascular endothelial growth factor receptor antibody or a T-cell receptor-like antibody.

10. The method for treating cancer of claim 7, wherein the pharmaceutical composition further comprises a biological probe, and the biological probe comprises a folic acid, a cell-penetrating TAT peptide, a fibroblast activation protein inhibitor, an arginylglycylaspartic acid peptide or a heparin polysaccharide.
